(19) United States
(12) Patent Application Publication
(10) Pub. No.: US 2022/0105109 A1
(43) Pub. Date: Apr. 7, 2022

(54) METHODS OF TREATING COVID-19 AND COMPOSITIONS THEREFOR

(71) Applicant: Barry E. Negrin, New York, NY (US)

(72) Inventor: Barry E. Negrin, New York, NY (US)

(21) Appl. No.: 17/113,163

(22) Filed: Dec. 7, 2020

Related U.S. Application Data
(60) Provisional application No. 63/086,832, filed on Oct. 2, 2020.

(51) Int. Cl. A61K 31/616; A61K 31/426

(52) U.S. Cl. CPC A61K 31/616; A61K 31/426

(57) ABSTRACT

Methods and compositions for treating COVID-19 in humans, particularly for treating at least one respiratory symptom, are disclosed. The composition includes a therapeutically effective amount of acetylsalicylic acid (ASA), expected to be in the range of approximately 0.4-4.0 grams per day. The composition may also include a therapeutically effective amount of famotidine, expected to be in the range of approximately 10-160 milligrams per day.

### RELATED APPLICATIONS

Priority is claimed from U.S. Provisional Patent Application No. 63/086,832 filed Oct. 2, 2020 and entitled “METHODS OF TREATING COVID-19 AND COMPOSITIONS THEREFOR”, the entirety of which is incorporated by reference herein.

### BACKGROUND OF THE INVENTION

### Field of the Invention

The invention is directed to methods of treating COVID-19 and compositions therefor generally, and in particular to methods of and compositions for treating respiratory symptoms and complications of COVID-19, especially in humans.

### Description of Related Art

Coronaviruses are a family of viruses that can cause illnesses such as the common cold, severe acute respiratory syndrome (SARS), and Middle East respiratory syndrome (MERS). In 2019, a new coronavirus was identified as the cause of a disease outbreak that is believed to have originated in China. The virus is now known as the severe acute respiratory syndrome coronavirus 2 (SARS-CoV-2). The disease it causes is called coronavirus disease 2019 (COVID-19). In March 2020, the World Health Organization (WHO) declared the COVID-19 outbreak a pandemic.

Angiotensin-converting enzyme 2 (ACE2) is a protein on the surface of many cell types. It is an enzyme that generates small proteins—by cutting up the larger protein angiotensinogen—that then go on to regulate functions in the cell. Using the spike-like protein on its surface, the SARS-CoV-2 virus binds to ACE2 prior to entry and infection of cells. ACE2 acts as a cellular doorway—a receptor—for the virus that causes COVID-19.

Angiotensin-converting enzyme (ACE) inhibitors are medications that help relax a person's veins and arteries to lower one's blood pressure. ACE inhibitors prevent an enzyme in the body from producing angiotensin IL, a substance that narrows blood vessels. This narrowing can cause high blood pressure and force the heart to work harder. Angiotensin II also releases hormones that raise blood pressure.

According to Garvin et al. (A mechanistic model and therapeutic interventions for COVID-19 involving a RAS-mediated bradykinin storm, eLife 2020;9:e59177, the entirety of which is incorporated by reference herein), once COVID-19 has established itself in the body, COVID-19 does not merely infect cells that already express numerous ACE2 receptors. Instead, it actively hijacks the body's own systems, tricking it into upregulating ACE2 receptors in places where they are usually expressed at low or medium levels, including the lungs.

The renin-angiotensin system (RAS) controls many aspects of the circulatory system, including the body's levels of bradykinin, which normally helps to regulate blood pressure. According to Garvin et al., when the virus affects the RAS, it causes the body's mechanisms for regulating bradykinin to go haywire. Bradykinin receptors are resensitized, and the body also stops effectively breaking down bradykinin. (ACE normally degrades bradykinin, but when the virus downregulates it, it cannot do this as effectively.)

The end result appears to be an enormous rise of bradykinin levels in the infected person. According to the bradykinin hypothesis, this “bradykinin storm” is responsible for many of COVID-19's lethal symptoms. Garvin et al. say in their paper that “the pathology of COVID-19 is likely the result of Bradykinin Storms rather than cytokine storms,” which had been previously identified in COVID-19 patients, but that “the two may be intricately linked.” Other papers had previously identified bradykinin storms as a possible cause of COVID-19's pathologies. Increased bradykinin levels could also account for other common COVID-19 symptoms. ACE inhibitors—a class of drugs used to treat high blood pressure—have a similar effect on the RAS system as COVID-19, increasing bradykinin levels. Garvin et al.: “the virus . . . acts pharmacologically as an ACE inhibitor . . . ”.

By acting like a natural ACE inhibitor, COVID-19 may be causing the same effects that hypertensive patients sometimes get when they take blood pressure-lowering drugs. ACE inhibitors are known to cause a dry cough and fatigue, two extremely common symptoms of COVID-19, which also include shortness of breath and/or difficulty breathing. Additionally, ACE inhibitors can increase blood potassium levels, which has also been observed in COVID-19 patients. According to Garvin et al., the similarities between ACE inhibitor side effects and COVID-19 symptoms support the bradykinin hypothesis.

This hypothesis is further supported by the fact that the insoluble by-products of ACE inhibitors are kinins (such as bradykinin) and are not properly filtered out of the blood. The kinins flow out of the kidneys and lodge themselves in the lungs' bronchial tubes. ACE inhibitor-induced coughing spells appear to represent the body's attempt to expel kinins from the lungs. Even after the drug is stopped, the cough can linger for months until all the kinins eventually find their way out of the lungs. In the case of COVID-19, bradykinin levels are thought to be significantly higher than those caused by ACE inhibitors, resulting in far worse respiratory symptoms (shortness of breath, difficulty breathing, hypoxia, etc.).

This hypothesis was also conjectured by Roche et al. in “A hypothesized role for dysregulated bradykinin signaling in COVID-19 respiratory complications”, FASEB J. 2020 May 2: 10.1096/fj.202000967, the entirety of which is incorporated by reference herein. In it, they hypothesize a role for dysregulated bradykinin signaling in COVID-19 respiratory complications and the potential benefit of bradykinin receptor blockers. SARS-CoV-2 is known to enter host cells in the respiratory system via the transmembrane protein, angiotensin converting enzyme 2 (ACE2). SARS-CoV-2 infection depletes ACE2 at the plasma membrane of infected cells. In the extracellular environment of both infected cells as well as neighboring bystander cells, ACE2 depletion increases the levels of des-Arg(9)-bradykinin (DABK), which is a bioactive metabolite of bradykinin (BK) that is associated with airway inflammation. SARS-CoV-2 infection severely affects host cell homeostasis by triggering endoplasmic reticulum stress, mitochondrial death signaling, downregulation of ACE2, upregulation of pro-inflammatory genes, and nuclear death signals, which ultimately lead to cell death. Cellular injury and inflammation induces BK-B1-receptor (B1R) upregulation and trafficking to the plasma membrane, which amplifies DABK-mediated inflammation and injury. Tissue injury and inflammation also increases BK levels and BK-B2-receptor (B2R) stimulation. Roche et al. hypothesize that ACE2 depletion in SARS-CoV-2-infected cells causes DABK accumulation in the extracellular environment of infected and neighboring bystander cells, which triggers a vicious positive feedback loop of inflammation and injury leading to even greater levels of DABK- and BK-mediated inflammation and injury.

It is an object of the invention to provide methods and compositions for treating COVID-19.

It is another object of the invention to provide methods and compositions for alleviating at least one symptom of COVID-19.

It is another object of the invention to provide methods and compositions for treating respiratory symptoms and complications of COVID-19 by at least partially alleviating the upsurge in bradykinin levels.

### SUMMARY OF THE INVENTION

The above and other objects are achieved by the invention, which are methods and compositions for treating COVID-19. In one embodiment, a method and composition for treating COVID-19 includes the step of administering a composition comprising a therapeutically effective amount of acetylsalicylic acid (ASA). The therapeutically effective amount of ASA is expected to be 0.4-4.0 grams per day. Preferably, the therapeutically effective amount of ASA is expected to be approximately 0.5-1.5 grams per day, equivalent to 1-3 “extra strength” over-the-counter doses of ASA. The ASA administration may be orally via tablet (swallowable, chewable, dissolvable, etc.), capsule, or liquid suspension. In addition or in the alternative, the ASA administration may be rectally in suppository form. In addition or in the alternative, the ASA administration may be via injection, e.g., intravenous, intramuscular, etc. In addition or in the alternative, the ASA administration may be performed transdermally. In addition or in the alternative, the ASA administration may be performed via inhalation. Optionally, the ASA administration may be performed in conjunction with other compounds.

In one embodiment, the invention is a method of treating at least one respiratory symptom of COVID-19 in humans. The method includes the step of administering a composition comprising a therapeutically effective amount of acetylsalicylic acid (ASA) to a human in need thereof. The expected therapeutically effective amount of ASA is approximately 0.4-4.0 grams per day and/or approximately 0.5-1.5 grams per day. Optionally, the administration step is performed orally. Optionally, the administration step is performed at least one of rectally, via injection, transdermally, or via inhalation. Optionally, the administration step further includes the step of dividing the therapeutically effective amount of ASA into a plurality of doses per day. Optionally, the administration step further includes the step of administering a therapeutically effective amount of famotidine to the human in need thereof. The expected therapeutically effective amount of famotidine is approximately 10-160 mg per day.

In another embodiment, the invention is a method of treating COVID-19 in humans. The method includes the step of administering a composition comprising a therapeutically effective amount of acetylsalicylic acid (ASA) to a human in need thereof. The expected therapeutically effective amount of ASA is approximately 0.4-4.0 grams per day and/or approximately 0.5-1.5 grams per day. Optionally, the administration step is performed orally. Optionally, the administration step is performed at least one of rectally, via injection, transdermally, or via inhalation. Optionally, the administration step further includes the step of dividing the therapeutically effective amount of ASA into a plurality of doses per day. Optionally, the administration step further includes the step of administering a therapeutically effective amount of famotidine to the human in need thereof. The expected therapeutically effective amount of famotidine is approximately 10-160 mg per day.

In another embodiment, the invention is a composition for treating at least one respiratory symptom of COVID-19 in humans and includes a therapeutically effective amount of acetylsalicylic acid (ASA). The expected therapeutically effective amount of ASA is approximately 0.4-4.0 grams per day and/or approximately 0.5-1.5 grams per day. Optionally, the therapeutically effective amount of ASA is divided into a plurality of doses per day. Optionally, the composition further includes a therapeutically effective amount of famotidine. The expected therapeutically effective amount of famotidine is approximately 10-160 mg per day.

### BRIEF DESCRIPTION OF THE DRAWINGS

None.

### DETAILED DESCRIPTION OF THE INVENTION

The action of COVID-19 functioning like an ACE inhibitor indicates that prophetically the worst of at least the respiratory symptoms (dry cough, shortness of breath, fatigue) may be treated by ASA. In several studies of ACE inhibitors, bradykinin was considered the likely culprit of the various side effects when taking the medication. See, e.g., Fox, A., Lalloo, U., Belvisi, M. et al., “Bradykinin-evoked sensitization of airway sensory nerves: A mechanism for ACE-inhibitor cough.”, Nat Med 1996;2, 814-817; McNally, E M, “Cough due to captopril.” West J Med 1987; 146:226-228, the entireties of both of which are incorporated by reference herein.

At least one study of treating ACE-inhibitor induced cough suggested that ASA can quickly and effectively reduce the problem. See Tenenbaum et al., “Intermediate but not Low Doses of Aspirin Can Suppress Angiotensin-Converting Enzyme Inhibitor-Induced Cough”, AJH 2000; 13:776-782, the entirety of which is incorporated by reference herein. In Tennenbaum et al., two different dosages of ASA were administered to patients with ACE-inhibitor induced cough: a “low” dose of 100 mg daily, and an “intermediate” dose of 500 mg daily. The 100 mg low doses of ASA were ineffective in suppressing ACE-inhibitor induced cough. However, the 500 mg intermediate doses of ASA were extremely effective, completely abolishing cough in five of 14 patients and significantly reducing cough in 13 of 14 patients. Significant reductions in both cough severity and cough frequency were observed with the 500 mg dose of ASA.

It is expected that at least relatively intermediate doses of ASA (e.g., approximately 400 mg) will alleviate the various COVID-19 issues believed to be caused at least in part by high levels of bradykinin in the body. This expected result is at least a little surprising in light of some COVID-19 literature, because some such literature cautioned against taking anti-pyretic agents such as NSAIDS, including ASA, when ill with COVID-19, as it was thought that a worse outcome may be experienced. See, e.g., “Covid-19: ibuprofen should not be used for managing symptoms, say doctors and scientists”, BMJ 2020;368:m1086; “Covid-19: European drugs agency to review safety of ibuprofen”, BMJ 2020;368:m1168.

In one anecdotal case, a 51-year-old non-smoking male in otherwise good health contracted COVID-19. The subject regularly takes 0.4 g ASA orally daily or twice daily for muscle aches and delayed onset muscle soreness (DOMS) as a result of frequent exercise and had taken at least 0.4 g ASA at least a few of the days during the approximately 14-day course of the illness. The subject exhibited none of the common respiratory symptoms and effects of COVID-19, i.e., no shortness of breath, no cough.

Therefore, given the Tennenbaum et al. results and the favorable result of the aforementioned anecdotal subject, it is expected that a therapeutically effective dose of ASA would begin in the range of approximately 400 mg ASA. However, some beneficial and therapeutic effect might also be realized with doses as small as 81 mg daily, currently commonly administered to forestall cardiac events. An expected upper range of a therapeutic dose of ASA for treating COVID-19 is approximately 4.0 g daily, which is the maximum usual adult dose of ASA for fever. In doses approaching 4.0 g daily, it is expected to break up the daily dose into several smaller doses periodically during the day, e.g., 400-800 mg every 4-6 hours.

It bears mentioning that the aforementioned anecdotal subject had also been taking 80 mg of famotidine daily for acid reflux. A number of studies suggest that COVID-19 patients who had been taking famotidine had better outcomes, e.g., fewer intubations, fewer deaths, milder symptoms, etc., than those who had not. Jiminez et al., “The influence of pH on SARS-CoV-2 infection and COVID-19 severity”, medRxiv preprint doi:10.1101/2020.09.10.20179135; Freedberg et al., “Famotidine Use Is Associated With Improved Clinical Outcomes in Hospitalized COVID-19 Patients: A Propensity Score Matched Retrospective Cohort Study”, Gastroenterology. 2020 September; 159(3): 1129-1131.e3; “Hartford Hospital Study: Pepcid, a Heartburn Medication, Helped Some COVID-19 Patients”, hartfordhealthcare.org/about-us/news-press/news-detail?articleid=28305&publicId=395, the entireties of which are incorporated by reference herein As a result, it is expected that the expected therapeutic doses of ASA will benefit from substantially concurrent doses of famotidine. Typical daily doses of famotidine range from 10-160 mg, and that dosage range is expected to be therapeutic and/or at least beneficial in treating COVID-19 in conjunction with 0.4-4.0 g of ASA. Since ASA tends to irritate some people's stomach linings, the administration of famotidine is expected to at least partially alleviate that potential side effect as well.

The invention is not limited to the above description. For example, while ASA is expected to be helpful and efficacious, it can be administered with other compounds that have demonstrated anti-COVID properties, including but not limited to famotidine, remdesivir, baricitnib, ibuprofen, interferon beta, convalescent plasma, dexamethasone, anticoagulants, monoclonal antibodies, and others now known or to be developed in the future. It is also expected that ASA will be combined with one or more typical pharmaceutical excipients, stabilizers, bulking agents, and the like as are known in the pharmaceutical industry. Some exemplary excipients are expected to include but not be limited to: cellulose, microcrystalline cellulose, hypromellose, polyethylene glycol, starch, surfactant(s), and the like.

Having described certain embodiments of the invention, it should be understood that the invention is not limited to the above description. Rather, the scope of the invention is defined by the claims appearing hereinbelow and includes any equivalents thereof as would be appreciated by one of ordinary skill in the art.

1. A method of treating at least one respiratory symptom of COVID-19 selected from the group consisting of dry cough, shortness of breath, or difficulty breathing in humans, the method comprising the step of administering a composition comprising a therapeutically effective amount of ASA to a human in need thereof, wherein the therapeutically effective amount of ASA is approximately 0.4-4.0 grams daily for multiple days.

2. (canceled)

3. A method of treating at least one respiratory symptom of COVID-19 selected from the group consisting of dry cough, shortness of breath, or difficulty breathing in humans according to claim 1, wherein the therapeutically effective amount of ASA is approximately 0.5-1.5 grams daily for multiple days.

4. A method of treating at least one respiratory symptom of COVID-19 selected from the group consisting of dry cough, shortness of breath, or difficulty breathing in humans according to claim 1, wherein said administration step is performed orally.

5. A method of treating at least one respiratory symptom of COVID-19 selected from the group consisting of dry cough, shortness of breath, or difficulty breathing in humans according to claim 1, wherein said administration step is performed at least one of rectally, via injection, transdermally, or via inhalation.

6. A method of treating at least one respiratory symptom of COVID-19 selected from the group consisting of dry cough, shortness of breath, or difficulty breathing in humans according to claim 1, said administration step further comprising the step of dividing the therapeutically effective daily amount of ASA into a plurality of doses per day.

7. A method of treating at least one respiratory symptom of COVID-19 in humans according to claim 1, said administration step further comprising the step of administering a therapeutically effective amount of famotidine to the human in need thereof.

8. A method of treating at least one respiratory symptom of COVID-19 in humans according to claim 7, wherein the therapeutically effective amount of famotidine is approximately 10-160 mg per day.

9. A method of treating COVID-19 in humans, the method comprising the step of administering a composition comprising a therapeutically effective amount of acetylsalicylic acid (ASA) to a human in need thereof.

10. A method of treating COVID-19 in humans according to claim 9, wherein the therapeutically effective amount of ASA is approximately 0.4-4.0 grams per day.

11. A method of treating COVID-19 in humans according to claim 9, wherein the therapeutically effective amount of ASA is approximately 0.5-1.5 grams per day.

12. A method of treating COVID-19 in humans according to claim 9, wherein said administration step is performed orally.

13. A method of treating COVID-19 in humans according to claim 9, wherein said administration step is performed at least one of rectally, via injection, transdermally, or via inhalation.

14. A method of treating COVID-19 in humans according to claim 9, said administration step further comprising the step of dividing the therapeutically effective amount of ASA into a plurality of doses per day.

15. A method of treating COVID-19 in humans according to claim 9, said administration step further comprising the step of administering a therapeutically effective amount of famotidine to the human in need thereof.

16. A method of treating COVID-19 in humans according to claim 15, wherein the therapeutically effective amount of famotidine is approximately 10-160 mg per day.

17. A composition for treating at least one respiratory symptom of COVID-19 in humans, comprising a therapeutically effective amount of acetylsalicylic acid (ASA).

18. A composition for treating at least one respiratory symptom of COVID-19 in humans according to claim 17, wherein said therapeutically effective amount of ASA is approximately 0.4-4.0 grams per day.

19. A composition for treating at least one respiratory symptom of COVID-19 in humans according to claim 17, wherein said therapeutically effective amount of ASA is approximately 0.5-1.5 grams per day.

20. A composition for treating at least one respiratory symptom of COVID-19 in humans according to claim 17, wherein said therapeutically effective amount of ASA is divided into a plurality of doses per day.

21. A composition for treating at least one respiratory symptom of COVID-19 in humans according to claim 17, further comprising a therapeutically effective amount of famotidine.

22. A composition for treating at least one respiratory symptom of COVID-19 in humans according to claim 21, wherein said therapeutically effective amount of famotidine is approximately 10-160 mg per day.
